(19) United States
(12) Patent Application Publication
(10) Pub. No.: US 2006/0051736 A1
(43) Pub. Date: Mar. 9, 2006

(54) Paraffin-control marker

(75) Inventors: Kevin Shields, Whitley Bay (GB); Eric Kanazawa, San Jose, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Applied Imaging Corp., San Jose, CA (US)

(21) Appl. No.: 11/202,584

(22) Filed: Aug. 11, 2005

Related U.S. Application Data
(60) Provisional application No. 60/603,042, filed on Aug. 19, 2004.

(51) Int. Cl. C12Q 1/00; G01N 1/30

(52) U.S. Cl. 435/4; 435/40.5

(57) ABSTRACT

A sample configured to be cut to form a set of serial sections. The sample includes a sample block; at least one tissue sample substantially embedded in the sample block; and a least one control-marker core substantially embedded in the sample block and having a select shape as viewed from an end of the control-marker core, wherein each serial section includes a cross section of the tissue sample and a cross section of the control-marker core, each cross section of the tissue sample is referred to as the tissue section and each cross section of the control-marker core is referred to as the control marker, and wherein each control marker has the select shape.

### CROSS-REFERENCES TO RELATED APPLICATIONS

This application claims priority to U.S. Provisional Patent Application No. 60/603,042, filed Aug. 19, 2004, titled “Paraffin-Control Marker,” of Kevin Shields and Eric Kanazawa, the disclosure of which is incorporated by reference herein in its entirety for all purposes.

### BACKGROUND OF THE INVENTION

The present invention relates generally to the analysis of samples, such as biological tissue samples that are chemically stained for protein-based markers, or have been processed for fluorescence in-situ hybridization (FISH), and more specifically to the use of control markers with serial sections, such that the control markers provide stain-control information and orientation information of serial sections mounted on slides.

Visual analysis of biological tissue samples often involves slicing the biological tissue samples into thin cross sections, often referred to as serial sections, to visualize structures of interest within the biological tissue sample. The serial sections are typically mounted on glass or plastic microscope slides to stabilize the serial sections and aid visualization. Visual analysis of mounted serial sections is often carried out by the naked eye (grossly) or by microscopy. In a typical sample preparation process, a tissue sample is dehydrated and embedded in paraffin to lend rigidity to the sample during slicing and mounting on microscope slides. Tissue samples are typically sliced into serial sections that are about 4-9 micrometers (μm) thick; however, other useful thicknesses are sliced. Once sliced, the serial sections are typically floated in water onto the microscope slides and moved into an appropriate location on the slides by a technician who physically manipulates the serial sections using, for example, a pair of tweezers or an artist's brush. Being relatively thin, the serial sections are relatively delicate and when placed on the microscope slides tend to deform by stretching, shrinking, being compressed, folding, flipping or a combination thereof. Moreover, the serial sections also tend to be placed on the microscope slides in rotated positions relative to one another. Such deformations and relative rotations often add to the difficulty in cross comparing serial sections. In some cases, such as FISH, it may be difficult to impossible to find sufficient common features to identify structures of interest between serial sections.

Serial sections of a tissue sample are typically cross-compared by histologists and pathologists, as well as others, to identify and locate the same tissue structure in the serial sections. For example, pathologists often cross compare serial sections that have been variously stained to aid in identifying and locating tissue structures of interest, such as groups of cancer cells or pre-cancerous cells. Stains of use have different affinities for different tissue structures and tend to color more intensely structures for which the stains have relatively high affinity. For example, a first serial section of a tissue sample is often stained with haematoxylin and eosin, referred to as H&E staining. Haematoxylin has a relatively high affinity for nuclei, while eosin has a relatively high affinity for cytoplasm. H&E stained tissue gives the pathologist important morphological and positional information about tissue structures of interest. For example, typical H&E staining colors nuclei blue-black, cytoplasm varying shades of pink, muscle fibers deep pinky red, fibrin deep pink, and red blood cells orange/red. The pathologist uses positional (e.g., color) information derived from the H&E stained tissue to estimate the location of corresponding tissue regions on successive serial sections of the tissue sample that are typically immunohistochemically stained. The successive serial sections may be immunohistochemically stained, for example, with HER-2/neu protein (a membrane-specific marker), Ki67 protein (a nuclei-specific marker), or other known stains. The use of such stains is well known in the art and will not be described in further detail.

Positional information derived from H&E stained serial sections is often crudely used to locate corresponding tissue on immunohistochemically stained serial sections. Pathologists commonly hold two or more slides up to a light and grossly attempt to judge the relative locations of structures of interest. As corresponding tissues may be distorted compared to the H&E section, and/or in a different location or orientation, position estimates may be many millimeters off, leading to poor and/or lengthy-repetitious analysis.

Poor and lengthy analysis arises not only in naked eye analysis of serial sections but also in computer-aided analysis of serial sections. Images of serial sections are often digitized and stored in a computer for computer-aided analysis. Present computer-aided analysis techniques do provide information for determining the distortions and relative rotations of serial sections captured in digital images of these sections. As a result of the distortion and relative rotations of a set of serial-images captured in digitized images, using location information derived from one serial-section image to locate structures in another serial-section image using computer-aided techniques is a laborious process fraught with misidentification and lengthy, repetitious analysis.

Accordingly, what is needed in the fields of pathology, histology, morphology, and other fields are new and useful apparatus and methods to simplify the cross comparison of serial sections. Also needed are new and useful apparatus and methods that provide improved orienting of serial sections relative to one another during cross comparison of the serial-sections either by naked-eye comparison or by computer-based comparison.

### BRIEF SUMMARY OF THE INVENTION

The present invention provides a set of serial sections that include tissue sections and control markers that are configured to have one or more select shapes to provide orientation information of serial sections mounted on slides to register the serial sections either manually or by a computer configured to recognize and register digital images of the control markers. In one embodiment the control markers are configured to stain as the tissue sections stain, such that the control markers provide stain-control information for each serial section.

In short, this is made possible by the generation of a sample that include a tissue sample and at least one control-marker core that has the select shape. According to one embodiment, a sample is provided from which a set of serial sections is configured to be cut. The sample includes a sample block; at least one tissue sample substantially embedded in the sample block; and a least one control-marker core substantially embedded in the sample block and having a select shape as viewed from an end of the control-marker core, wherein each of at least two serial sections includes a cross section of the tissue sample and a cross section of the control-marker core, each cross section of the tissue sample is referred to as the tissue section and each cross section of the control-marker core is referred to as the control marker, and wherein each control marker has the select shape.

According to a specific embodiment, the select shape is at least one of a square, a triangle, and a circle. According to another specific embodiment, if one of the serial sections is distorted, rotated, or flipped, the control marker associated with this serial section is configured to substantially similarly distort, rotate, or flip.

According to another specific embodiment, The control markers are configured to stain as the serial sections associated with the control markers stain to provide stain control information for the serial sections. The sample block might be formed of paraffin, agar, or resin. The control markers include at least one of tissue, such as a control-cell line, a tissue-like substance, a fluorescent material, ink, dye, and a condensed material.

According to another embodiment, a method for forming a set of serial sections includes forming, in a paraffin block, at least one hole that has a select shape; filling the hole with a marker substance and paraffin; and slicing the paraffin block to form the set of serial sections, wherein each of the serial sections includes a cross section of the tissue sample and a cross section of the marker substance, the cross sections of the tissue sample are referred to as the tissue sections, the cross sections of the marker substance are referred to as the control markers, and wherein each control marker has the select shape.

The step of forming the hole might include boring the hole with a needle that has the select shape. The needle may be configured to be manually operated or operated by a tissue microarrayer. According to a specific embodiment, the method further includes mounting the serial sections respectively on a set of slides, wherein if at least one of the serial sections during mounting is distorted, rotated, or flipped, the control marker associated with this serial section is substantially similarly distorted, rotated, or flipped. According to another specific embodiment, the method further includes registering the select shapes of at least two of the control markers to register the tissue sections associated with these control markers. According to yet another embodiment, the method further includes staining at least one of the serial sections, wherein the control marker associated with this serial section is configured to stain a select color, wherein the color the control marker stains is an indicator of whether this serial section correctly stains.

According to another embodiment, a computerized method is provided for registering a plurality of serial-section images that include tissue-section images and control-marker images. The method includes performing pattern recognition on the control-marker images, wherein the control marker images have one or more select shapes; and registering the select shapes of the control-marker images to register the tissue-section images. The registering step might be displayed on a display of the computer. The step of registering might further include at least one of shearing, skewing, rotating, and flipping at least one of the control-marker images to corresponding shear, skew, rotating, and flip the serial-section image associated with this control-marker image.

A further understanding of the nature and advantages of the present invention may be realized by reference to the remaining portions of the specification and the drawings.

### BRIEF DESCRIPTION OF THE DRAWINGS

FIG. 1 is a time ordered sequence of events of a tissue sample, which is embedded in a paraffin block, sliced into a set of serial sections that are respectively mounted on a set of slides;

FIG. 2 is a simplified schematic of the set of serial sections shown in further detail;

FIG. 3 is a perspective view of a paraffin block that includes a set of control-marker cores and an embedded tissue sample according to one embodiment of the present invention;

FIG. 4 is a simplified end view of two control-marker cores that include a plurality of marker layers according to one embodiment of the present invention;

FIG. 5 is a simplified schematic of a control-marker core according to another embodiment of the present invention;

FIG. 6 is a simplified schematic of a set of serial sections that might be cut from the paraffin block shown in FIG. 3;

FIG. 7 is a simplified schematic of two serial sections that are approximately registered via the approximate registration of the serial sections' control markers;

FIG. 8A is a simplified diagram of a paraffin block that includes a tissue sample and a set of gradient control-marker cores;

FIG. 8B is a simplified schematic of a set of serial sections that might be cut from paraffin block that is shown in FIG. 8A;

FIG. 9 is a simplified schematic of a system that is configured to generate, store, and process digital images of serial sections according to an embodiment of the present invention;

FIG. 10 is a simplified schematic of a set of serial-section images displayed on display of the system that is shown in FIG. 9;

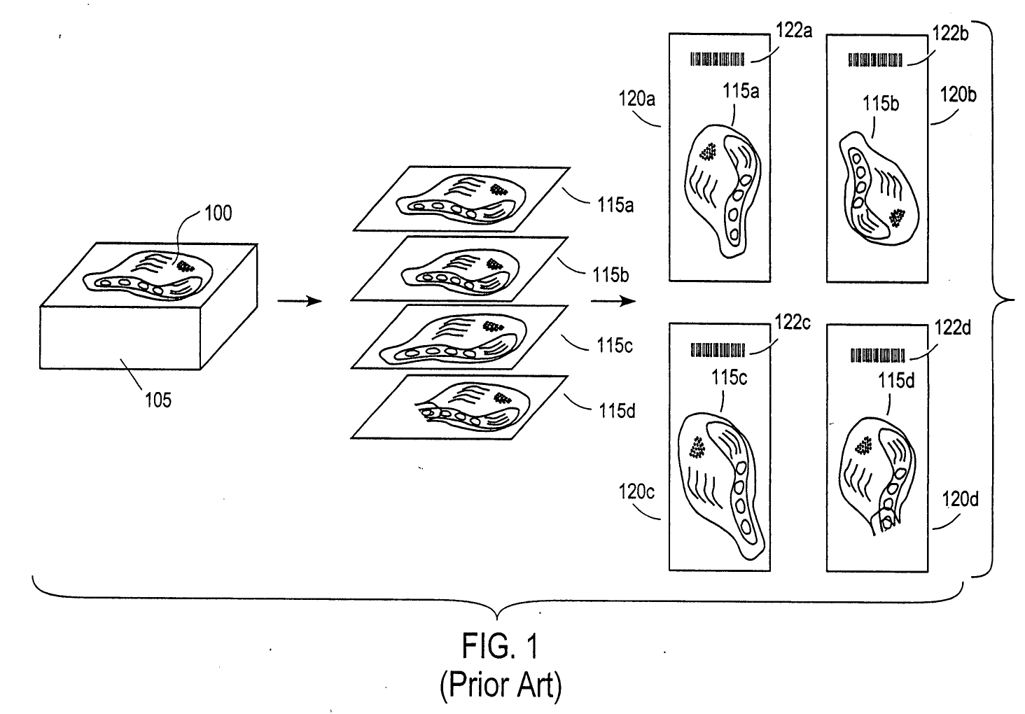

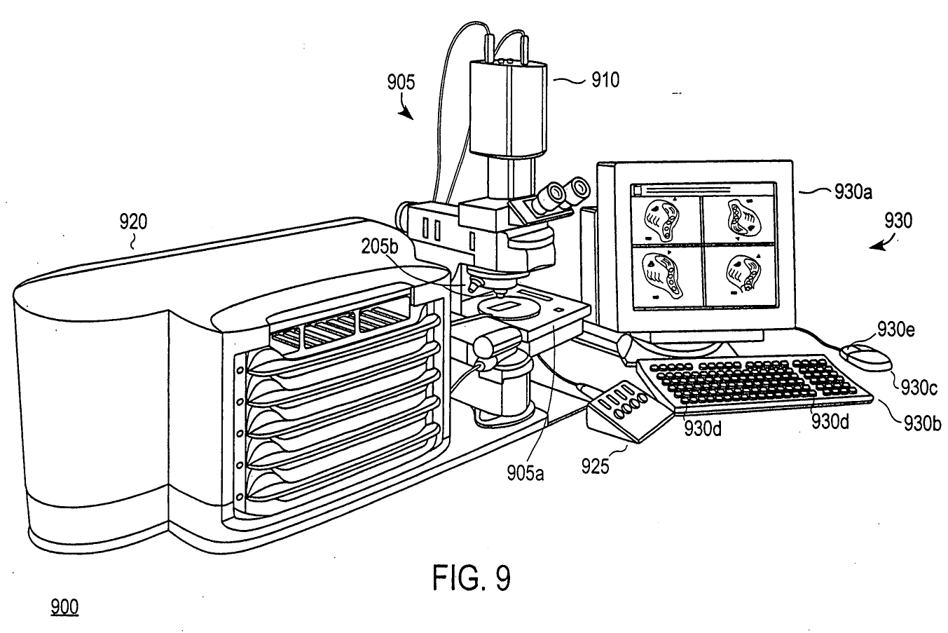

FIGS. 1A-11F show a time ordered sequence of events of the approximate registration of a set of control-marker images by the system that is shown in FIG. 9 according to an embodiment of the present invention;

FIG. 12 is a simplified schematic of a ghost image whose transparency may be adjusted using a slider bar;

FIG. 13 is a simplified schematic of a coordinate system imposed over a set of serial-section images that are displayed on the display;

FIG. 14 is a simplified schematic of a reference-link region positioned over a ghost image;

FIG. 15 is a simplified schematic of the reference-link region and shows a set of arrows that indicate various directions a handle of the reference-link region might be moved to skew, shear, and/or rotate the reference-link region and the ghost image;

FIG. 16 is a simplified schematic of a paraffin block having a plurality of holes formed therein;

FIG. 17 is a simplified schematic of a tissue microarrayer that is configured to extract paraffin cores from a paraffin block;

FIG. 18 is a simplified schematic of a paraffin block that includes a set of PCMCs (paraffin-control-marker cores, shown in phantom) that respectively include a set of control-marker cores according to an embodiment of the present invention;

FIG. 19 is a simplified schematic of a mold that includes a tissue sample, a pair of PCMCs, and control-marker cores associated with the PCMCs, such that paraffin might be poured into the mold to form a paraffin block;

FIG. 20 is a simplified schematic paraffin block having a set of holes formed therein that are configured to respectively receive a set of PCMCs;

FIG. 21 is a simplified schematic of a TMA (tissue microarray) that includes a plurality of tissue samples disposed in the array and a pair of PCMCs disposed at opposite corners of the array;

FIG. 22 is a simplified schematic of a set of serial sections that are cut from the TMA;

FIG. 23 is a high-level flow chart having steps for generating a set of serial sections that include tissue sections and control markers that have select shapes, such that the control markers are configured to provide positional information of the serial sections mounted on a set of slides; and

FIG. 24 is a high-level flow chart having computerized steps for substantially registering a plurality of control-marker images that have selects shapes and that are respectively included in a plurality of serial-section images, such that the substantial registration of the shapes of the control-marker images provides for substantial registration of the serial-section images and tissue-section images that might be included in the serial-section images.

### DESCRIPTION OF SPECIFIC EMBODIMENTS

Overview

The present invention provides a paraffin block having one or more control markers that are configured to provide control information for variously stained cross-sectional slices of a tissue sample, and provide orientation information for the cross-sectional slices, which are mounted on slides, relative to one another.

Particular applications of the present invention are in the fields of pathology, and other medical or bioscience fields, to provide quality control for staining processes, provide relative orientation information for mounted cross-sectional slices of a tissue sample (typically referred to as serial sections), correct for distortion and relative rotations between digitized images of serial sections as well as other applications. A first serial section of a tissue sample, often used as a reference section, is typically stained with haematoxylin and eosin, and is commonly referred to as an H&E section. Subsequent serial sections of the tissue sample are often immunohistochemically stained with markers to color and aid in locating structures of interest, such as cancerous and pre-cancerous cells. Known immunohistochemical stains include, for example, HER-2/nue protein, Ki67 protein, ER, and PgR.

Paraffin is commonly used to lend rigidity to tissue samples for slicing and mounting. A tissue sample is typically embedded in a paraffin block by placing the tissue sample in a mold and pouring warm paraffin into the mold to embed the tissue sample. Once the paraffin is cooled, the formed paraffin block and tissue sample may be sliced to form a set of serial sections. The serial sections are typically floated in a water bath onto slides for staining and analysis. The serial sections are often stained with various protein specific markers to provide improved visualization of tissues of interest in the serial sections. Tissues of interest might include precancerous cells, cancerous cells and the like. Subsequent to staining, the serial sections are cross compared to analyze the tissues of interest in the serial sections. Cross comparison is often hampered by distortions, rotations, and flipping of the serial section that occur during slicing, mounting, and processing (e.g., paraffin removal, staining, etc.) of the serial section.

FIG. 1 is a time ordered sequence of events of a tissue sample 100 that is embedded in a paraffin block 105, and is sliced into a set of serial sections 115 that are mounted respectively on a set of slides 120. Slides 120 might include a respective set of bar codes 122 or the like for identifying and cataloging the serial sections. The mounted serial sections are variously distorted and rotated with respect to one another.

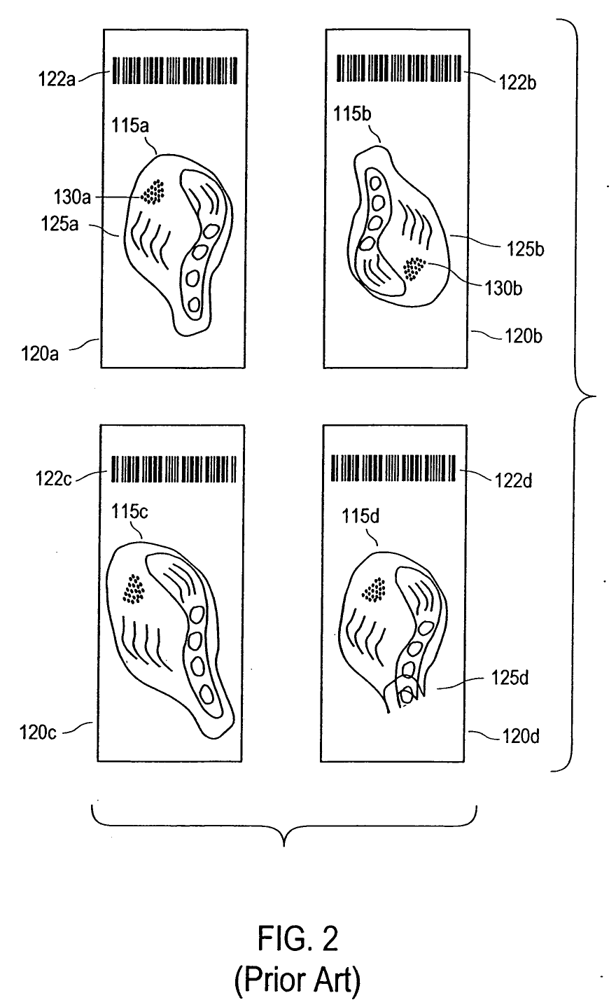

FIG. 2 is a further detailed view of serial sections 115 and shows the serial sections variously distorted and rotated with respect to one another. For example, serial section 115b is shown to be compressed in region 125b as compared with the corresponding region 125a of serial section 115a. Serial section 15b is also shown to be vertically compressed relative to serial section 115a. In addition to being compressed, serial sections may also be stretched. For example, serial section 115c is shown to be stretched along its longitudinal axis. Serial section 115c is also shown to be rotated relative to the other serial sections, and serial section 115d is shown to have a folded portion 125d at a bottom end of the serial section. As described briefly above, deformations and relative rotations of serial sections often make cross comparisons between serial sections relatively difficult. For example, location information of a structure 130a derived from serial section 115a may provide limited help in locating the corresponding structure 130b in serial section 115b as structures 130a and 130b are in different relative locations within their respective serial sections as a result of compression in region 125b of serial section 115b. This and other cross comparison difficulties are addressed by embodiments of the present invention.

Control Markers and Paraffin Blocks

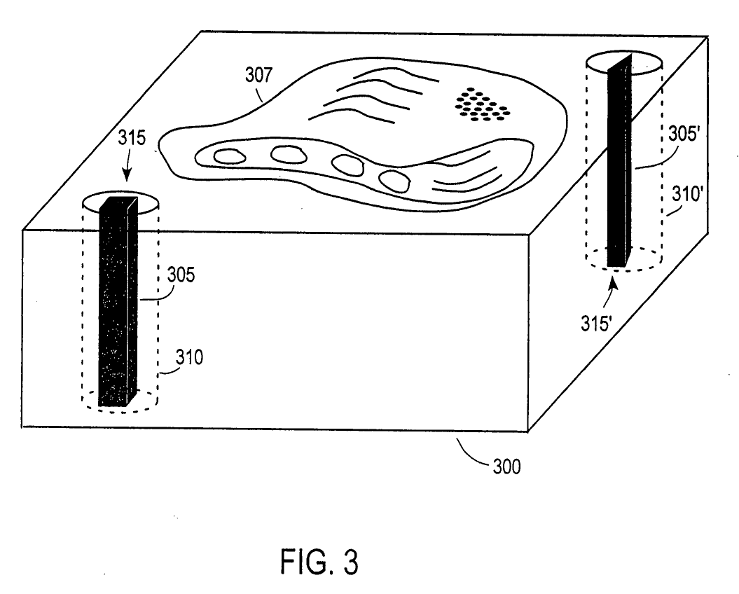

FIG. 3 is a perspective view of a paraffin block 300 that includes a first control marker-core 305, a second control-marker core 305′, and an embedded tissue sample 307 according to one embodiment of the present invention. For convenience, control-marker cores 305 and 305′ are described in detail first, paraffin block 300 is described second, and thereafter, a set of serial sections that might be sliced from a paraffin block are described. Control-marker cores 305 and 305′ may extend from a top surface of the paraffin block to a bottom surface of the paraffin block. Alternatively, the control-marker cores might be embedded in the paraffin block. According to one embodiment, control-marker cores 305 and 305′ are respectively embedded in paraffin cores 310 and 310′. For convenience, portions of the paraffin cores that extend into paraffin block 300 are shown in phantom. Each combined control-marker core and paraffin core is referred to herein as a paraffin-control-marker core (PCMC). The PCMC that includes control-marker core 305 and paraffin core 310 is labeled with the reference numeral 315, and the PCMC that includes control-marker core 305′ and paraffin core 310′ is labeled with the reference numeral 315′. According to one embodiment, PCMCs 315 and 315′ are inserted in the paraffin block after the paraffin block is formed. Insertion of PCMCs into paraffin blocks is described in detail below.

Control-marker cores have select shapes as viewed from the ends of the control-marker cores. For example, control-marker core 305 has a rectangular shape, and control-marker core 305′ has a triangular shape. Control-marker cores might have other shapes such as circular, an arbitrary and capricious shape or the like. Control-marker cores may be formed from one or more marker substances, such as tissue (e.g., a control tissue, such as a controlled-cell line), a tissue-like substance, a fluorescent material (e.g., one or more types of pollen grains), ink or dye (e.g., a dye that is not soluble in water, alcohol or the like), or a condensed material (e.g., plastic, resin, fibers, etc.). The foregoing list of marker substances is illustrative and not exclusive of the various marker substances that might be used to form a control marker. On review of the instant description, figures, and claims, those of skill in the art will recognize other marker substances that might be used to form a control-marker core. While paraffin block 300 is shown and described as including two control-marker cores, paraffin blocks according to alternative embodiments of the present invention, may include one or more control-marker cores.

According to one embodiment, control-marker cores include a substantially homogeneous distribution of one or more marker substances. According to an alternative embodiment, control-marker cores have layers of marker substances.

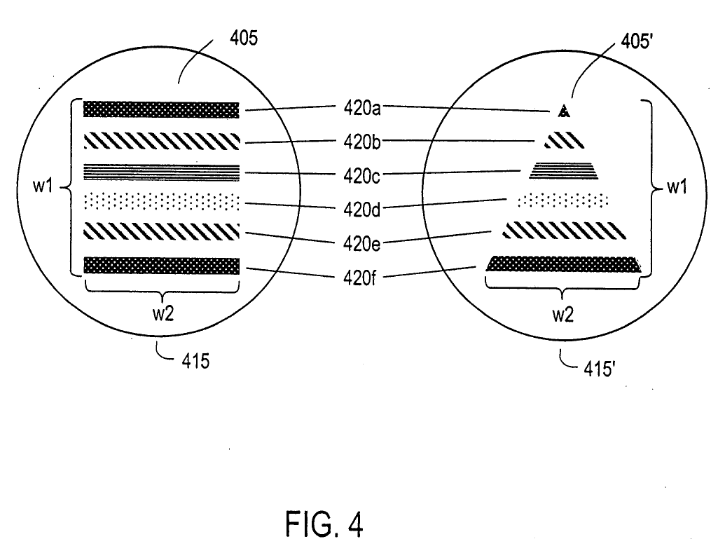

FIG. 4 is a simplified end view of a PCMC 415 and a PCMC 415′ that, respectively, include a control-marker core 405 and a control-marker core 405′. Control-marker cores 415 and 415′ each include a plurality of control-marker layers 420 according to one embodiment of the present invention. Each control-marker layer is labeled with the base reference numeral 420 and an alphabetic suffix. The control-marker layers might be formed from one or more marker substances, which are described above. For example, control-marker layers 420a and 420f might be formed from tissue, such as a controlled-cell line. The controlled-cell line might provide a stain control for a serial section staining process (described in further detail below). Further, control-marker layers 420b and 420e might be formed from a fluorescent material, such as pollen grains, ink, dye, etc. The fluorescent material might be configured to fluoresce under one or more wavelengths used for FISH (fluorescence in situ hybridization) analysis (also referred to in the art as chromosome painting). Further yet, control-marker layers 420c-420d might be formed from a visible-tissue-like substance. The foregoing described marker layers are illustrative of an embodiment of the present invention as the marker layers may be formed from a variety of other substances, such as those substances described above. While control-marker cores 405 and 405′ are each shown to include six control-marker layers, other control-marker cores might include fewer or more then six control-marker layers.

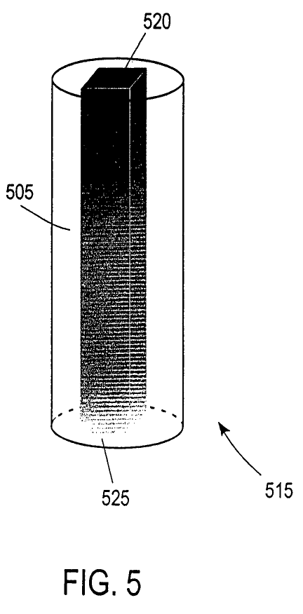

FIG. 5 is a simplified schematic of a PCMC 515 that includes a control-marker core 505 that has a gradient density according to one embodiment of the present invention. Control-marker core 505 includes a marker-substance (e.g., control tissue) that has a gradient density that changes from high to low along the length of the control-marker core. The marker substance has a highest density at a first end 520 of the control-marker core, and the density decreases along the control-marker core toward a second end 525 of the control-marker core. While control-marker core 505 is shown as including a marker substance that has a density that decreases from one end of the control-marker core to the other end of the control-marker core substantially linearly, other control-marker cores might include marker substances whose densities' vary periodically, randomly, according to a mathematical function (e.g., exponentially, logarithmically, etc.), or the like.

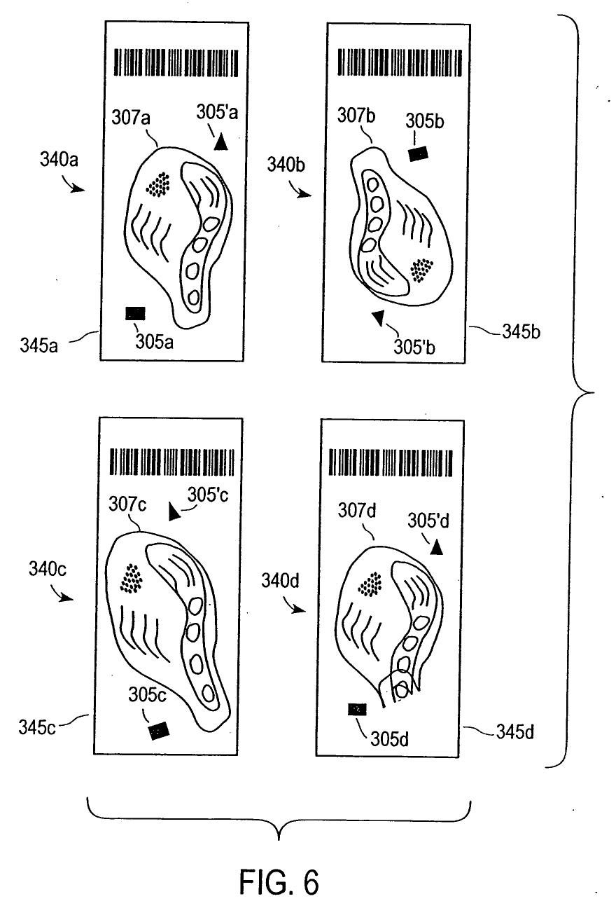

FIG. 6 is a simplified schematic of a set of serial sections 340 that might be cut from paraffin block 300 according to one embodiment of the present invention. Each serial section is labeled in FIG. 6 with the base reference number 340 and an alphabetic suffix. Each serial section includes a cross section of tissue sample 307 and cross sections of control-marker cores 305 and 305′. The cross sections of the tissue sample are referred to as “tissue sections,” and the cross sections of the control-marker cores are referred to herein as “control markers.” The control markers are labeled with the base reference numerals of their associated control-marker cores and alphabetic suffixes. The set of serial sections 340 might be mounted, respectively, on a set of slides 345. Once mounted on the slides, the paraffin from the paraffin block and the paraffin cores might be removed from the serial sections.

According to one embodiment, control markers 305a-305d and 305′a-305′d (or select layers thereof, e.g., if the control markers are layered as shown in FIG. 4) are configured to stain as serial sections 340 are stained. For example, control markers 305a-305d and 305′a-305′d might include one or more controlled cell lines that might be configured to be stained by a controlled amount according to the particular stain applied to the serial sections. Further, the control markers might stain different colors by different stains. For example, the control markers might be configured to stain blue-black by H&E stain, stain pink by HER-2/neu protein, stain orange by ER stain, and stain red by Ki67 protein. As the control markers are configured to stain (e.g., different colors by different stains), the control markers are configured to serve as stain controls to indicate whether a serial section has been properly stained. More specifically, each control marker of each serial section provides a quality control check that the serial section has been stained properly. That is, each serial section on each slide has its own control that indicates whether the serial section is properly stained. For example, if a control marker that is configured to stain blue-black by H&E stain, does not stain, stains the wrong color, or stains by an incorrect amount, these staining errors may indicate that something has gone wrong in the staining process. For example, a stain error (e.g., color error) of the control markers in an H&E staining process might indicate that the H&E stain has broken down, has otherwise been fouled, or might indicate other error. Therefore, any data that is collected from the stained tissue section might be suspect as bad data.

According to a further embodiment, the control markers 305a-305d and 305′a-305d might be configured to stain different colors by a given stain. For example, control markers 305a-305d might be configured to stain blue-black by H&E stain, whereas control markers 305′a-305′d might be configured to stain light pink by the H&E stain. For example, control markers 305a-305d might be configured to be stained by haematoxylin in the stain (e.g., control marker 305a-305d might include a control nuclei tissue), wherein as control markers 305′a-305′d might be configured to be stained by the eosin in the stain (e.g., control markers 305′a-305′d might include a control cytoplasm tissue). Disparate staining of the control markers in serial sections provides further quality control for various staining processes.

As serial sections are sliced from a paraffin block, mounted on slides, and processed, the serial sections often deform by being stretched, compressed or the like. The serial sections also often tend to rotate and/or flip (e.g., flipped front to back) during mounting. According to one embodiment, the control markers are configured to stretch, compress, rotate, and/or flip as their associated serial section stretch, compress, rotate, and flip.

For example, as shown in FIG. 6, tissue section 307c is shown as having been stretched along its longitudinal axis. The control markers associated with tissue section 307c, namely control markers 305c and 305′c, are stretched similarly to tissue section 307c. Not only are the control markers similarly stretched, the control markers also maintain their relative positions with respect to the tissue sections. For example, as tissue section 307c is stretched, and the top and the bottom of the tissue section are moved apart from one another; control markers 305c and 305′c (located proximate to the top and bottom of the tissue section) similarly move apart from one another, but maintain their relative positions with respect to the tissue section. Stated alternatively, each serial section, including its tissue section and its control marker, tends to deform, rotate, and flip in continuum. Therefore, the positions of control markers on a slide substantially memorialize the cumulative deformations, rotations, and flipping of their associated serial sections. Therefore, by observing control markers of the serial sections, one may relatively easily determine whether one or more serial sections have been deformed, rotated, and/or flipped. And the positions of the control markers may be used as position references to relatively easily rotate and/or flip the serial sections (i.e., flip the serial sections' slides) to approximately register the control markers, and thereby, approximately register the tissue sections associated with the control markers.

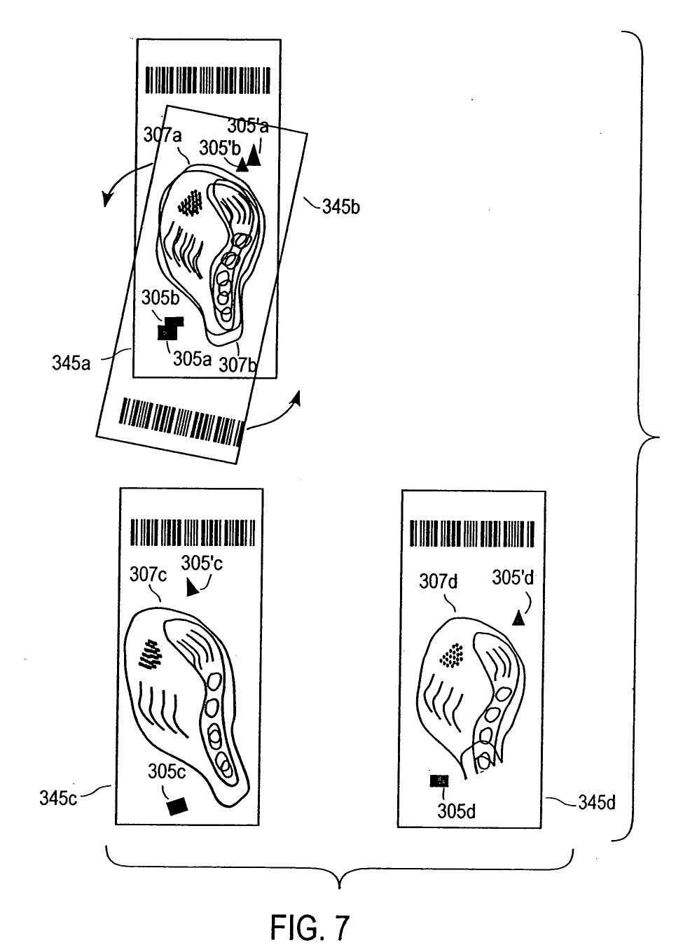

FIG. 7 is a simplified schematic of serial sections 340a and 340b being approximately registered (e.g., by hand) via the approximate registration of control markers 305a and 305b, and 305′a and 305′b.

Control markers might have widths that provide for relatively easy visualization grossly or using computer-aided analysis. The widths of control markers are indicated by w1 and w2 in FIG. 4. Control markers, according to one embodiment of the present invention, have widths of approximately 1 millimeter to approximately 5 millimeters, inclusive. According to other embodiments, control markers might have widths less than 1 millimeter or greater than 5 millimeters. As the control markers have widths that are relatively easily visualized, during cross comparison of serial sections, one can determine relatively quickly whether the serial sections have been deformed, rotated, and/or flipped during processing and orient the serial section to approximately register the control markers, and thereby, to approximately register the serial sections.

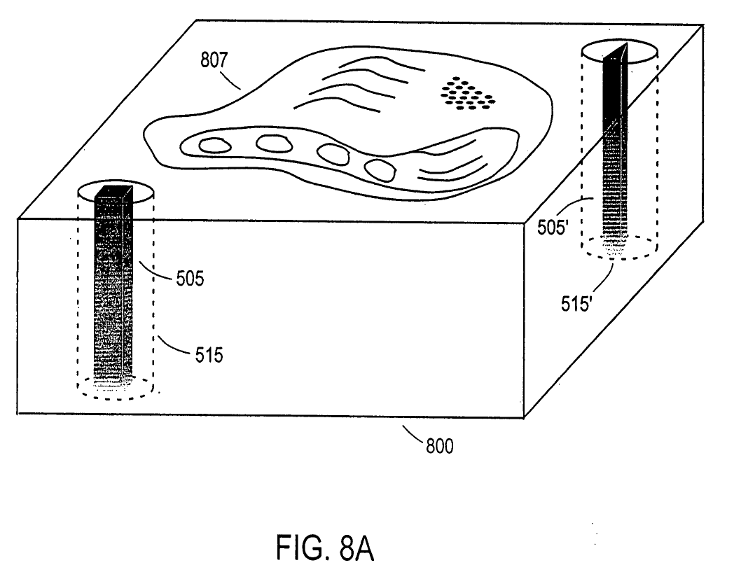

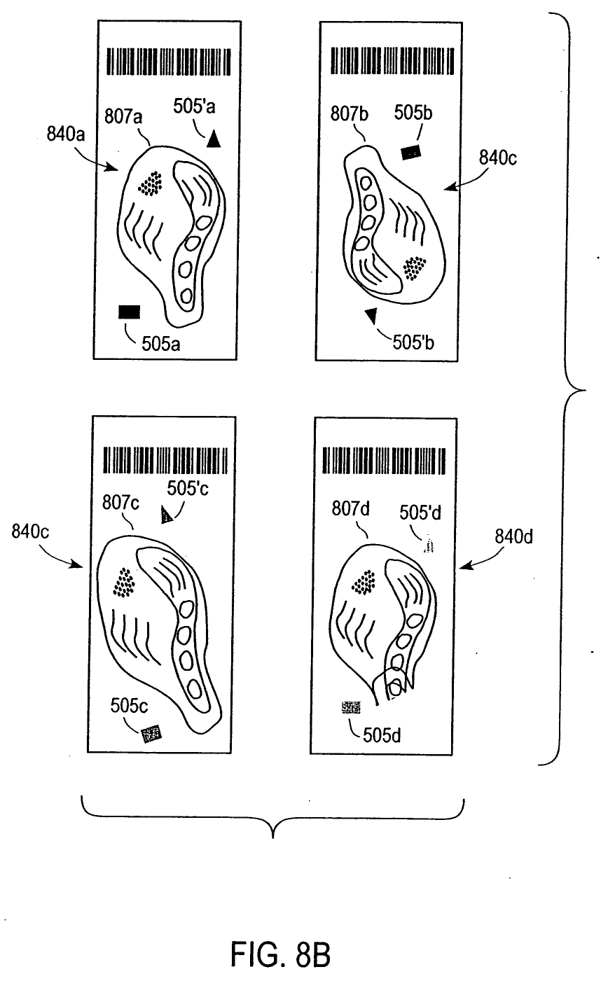

FIG. 8A is a simplified schematic of a paraffin block 800 that includes a tissue sample 807 and PCMCs 515 and 515′ according to one embodiment of the present invention. PCMCs 515 and 515′ respectively include control-marker cores 505 and 505′ that have gradient densities. The gradient densities of both control-marker cores 505 and 505′ decrease from the top of the paraffin block to the bottom of the paraffin block. While the gradient densities of control-marker cores 505 and 505′ decrease from the top of the paraffin block to the bottom of the paraffin block, the gradient densities of these control-marker cores might be opposite from one another. That is, one gradient density might decrease, and the other gradient density might increase, from the top of the paraffin block to the bottom of the paraffin block. FIG. 8B is a simplified schematic of serial sections 840a-840d that might be cut from paraffin block 800. The sets of control markers 505a and 505′a, 505b and 505′b, 505c and 505′, and 505′d and 505′d have different densities that provide position information for the serial section with respect to uncut tissue sample 807 and with respect to one another. For example, serial section 840a having control markers 505a and 505′a that are relatively dark (i.e., a relatively high density control marker) will have been cut from a relatively higher position of tissue sample 507 than serial section 840d having control markers 505d and 505′d that are relatively light (i.e., a relatively low density control marker). Accordingly, one viewing control markers 505′ and 505′a, and 505′d and 505′d will be able to determine relatively easily the relative positions of sections 807a and 807d with respect to uncut tissue sample 807. Digital Image Correction of Serial-Section Images having Control-Marker Images

FIG. 9 is a simplified schematic of a system 900 that is configured to generate, store, and process digital images of serial sections, such as digital images of serial sections 340a-340d, according to an embodiment of the present invention. For convenience, digital images of serial sections are referred to as serial-section images. System 900 is further configured to register two or more serial-section images to provide for relatively simplified cross comparison of the serial-section images and/or for optical processing of the serial-section images by the system. Prior to discussing registration of serial-section images by system 900, various components of system 900 are described.

According to one embodiment, system 900 is the ARIOL SL-50™ system manufactured by Applied Imaging Corporation, owner of the present invention. System 900 includes a microscope 905 with an attached camera 910, a slide loader 920, a stage manipulator 925, and a computer 930.

Microscope 905 magnifies images of the serial sections, usually, but not necessarily, one at a time, for ocular display and for image capture by camera 910. Microscope 905 is configured to magnify images of the serial sections at variety of magnifications, such as, but not limited to, 1.25×, 5×, 10×, 20×, and 40×. According to one embodiment, microscope 905 is a BX-61™ microscope manufactured by Olympus America, Inc. According to one embodiment, camera 210 is a 4912 CCIR™ camera manufactured by COHU, Inc. and has a 752×582 active-CCD-pixel matrix. The active-CCD-pixel matrix digitizes images of serial sections for delivery to computer 930.

Slide loader 920 is an automated device for delivery and removal of microscope slides to and from the microscope's stage 905a, which positions the slides under the microscope's objectives 905b for magnification. According to one embodiment, slide loader 920 holds up to 50 microscope slides, which can be randomly accessed for delivery to stage 905a. According to one embodiment, slide loader 920 is an SL-50™ Random Access Slide Loader manufactured by Applied Imaging Corporation.

According to one embodiment, computer 930 is a dual processor personal computer having two Intel XEON™ 1.8 gigahertz microprocessors and runs WINDOWST™ XP PROFESSIONAL™ operating system. The computer includes a display 930a, input devices 930b and 930c, and a memory device (not shown). Display, as referred to herein, includes any device capable of displaying digital images such as a CRT, a liquid crystal display, a plasma display or the like. Input device, as referred to herein, includes any device capable of generating computer input including, but not limited to, a mouse, trackball, touchpad, touchscreen, joystick, keyboard, keypad, voice activation and control system, or the like. The memory device includes any memory that is capable of storing and retrieving digital images and includes, but is not limited to, one or a combination of, a hard drive, floppy disk, compact disk (CD), digital videodisk (DVD), ROM, EPROM, EEPROM, DRAM, SRAM, or cache memory. While the forgoing describes equipment and software included in a particular embodiment of the present invention, those of skill in the art will recognize that various substitutes and alternatives may be included in system 900 without deviating from the spirit of the present invention.

The functionality of the specific embodiment is to provide digitized images for display so that a user can examine and manipulate the images. Computing and display technologies are ever evolving, and the invention does not require any specific type or configuration of computer. In addition, while the specific embodiment uses a CCD (charged coupled device) camera to digitize the magnified images of the serial sections, the invention does not require any specific type of digitizing mechanism. Cameras using other imaging array technology, such as CMOS, could be used, or the magnified slide image could be captured on photographic film, and the photographic film could be scanned in order to digitize the images. Further, as described in U.S. patent application Ser. No. 10/165,770, filed Jun. 6, 2002, and published Jan. 16, 2003 as Published Patent Application No. 2003/0012420 A1 to Nico Peter Verwoerd et al., microscope slides can be digitized using a high-resolution flatbed scanner and the digital images of the slides generated thereby may be loaded into computer 930.

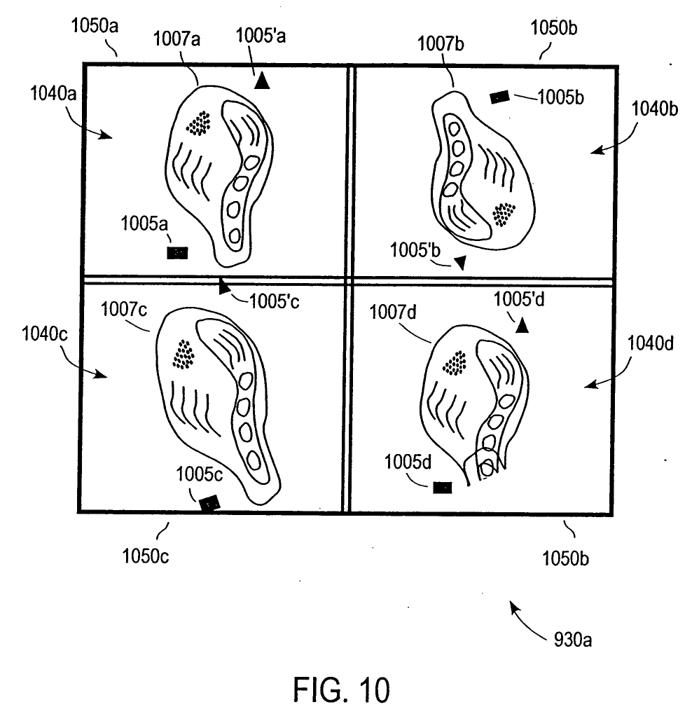

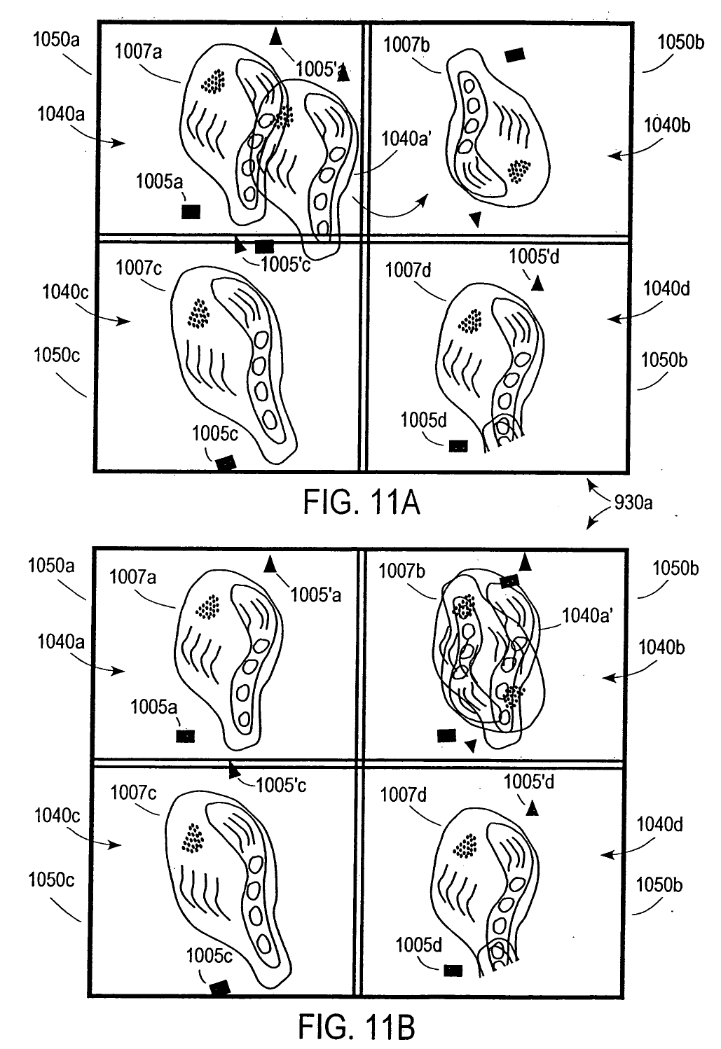

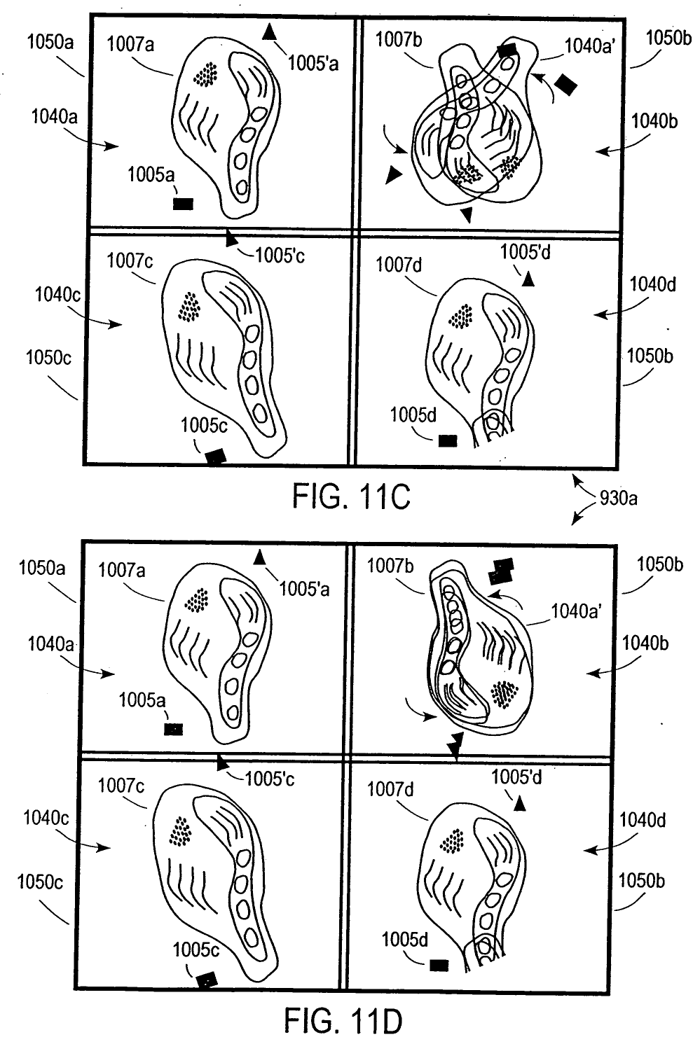

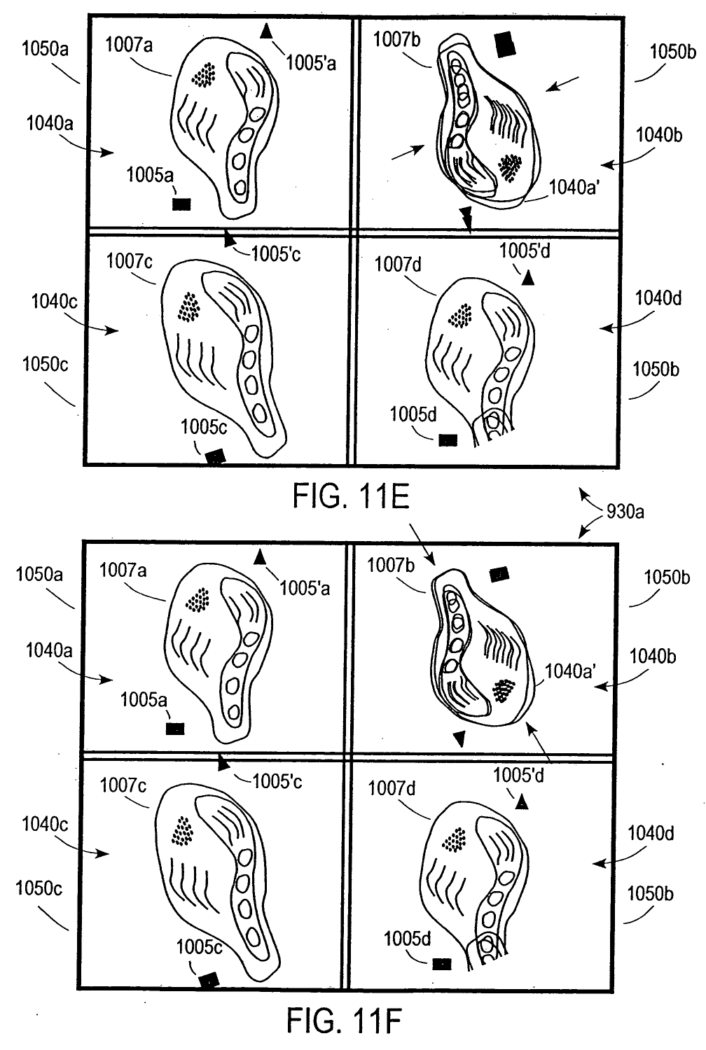

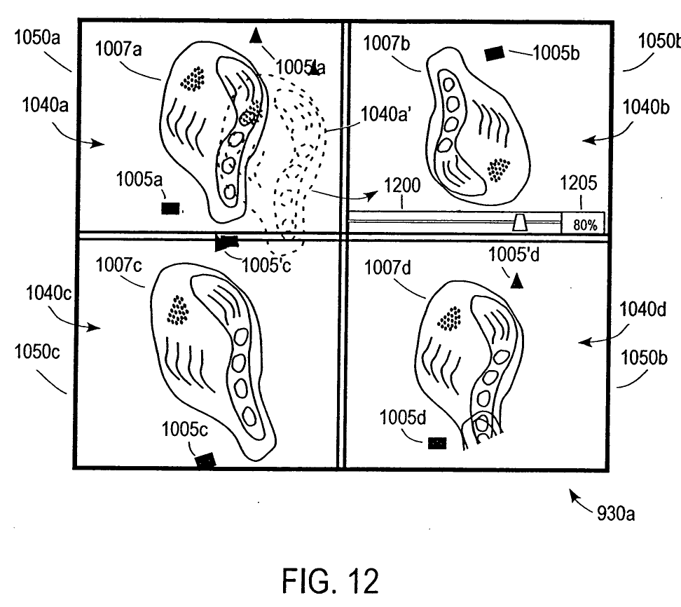

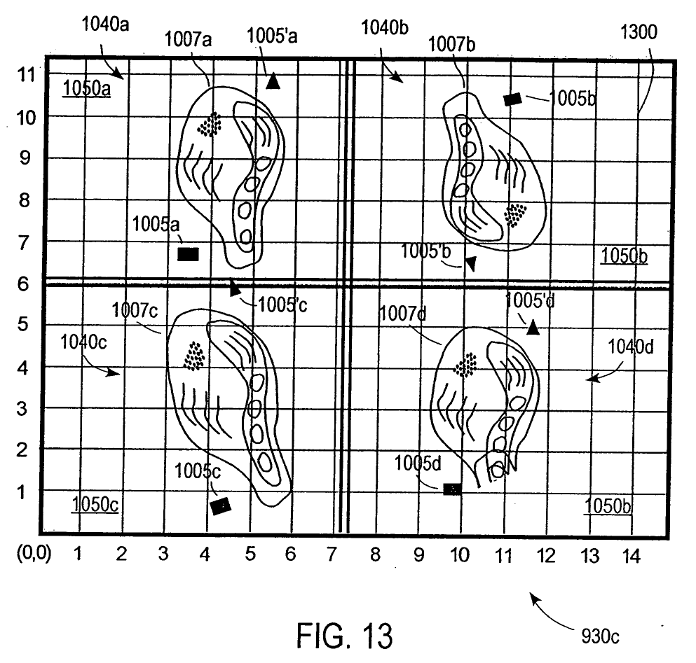

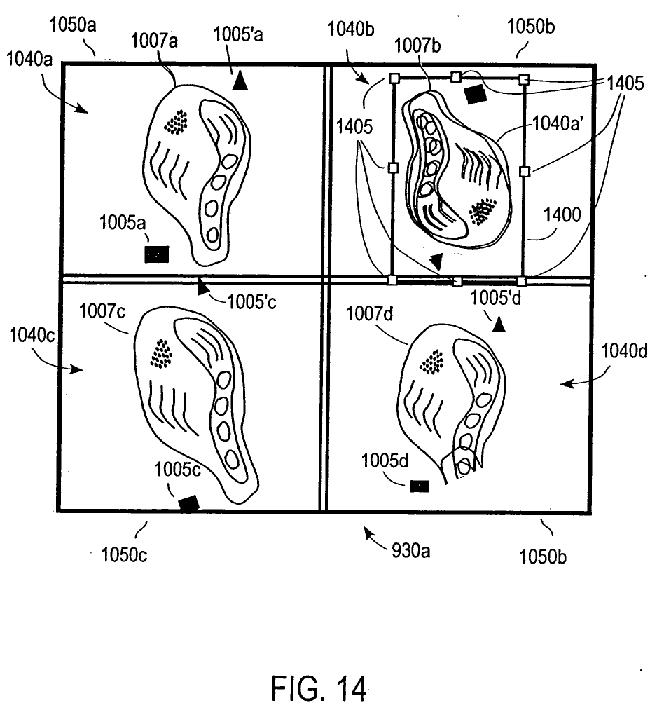

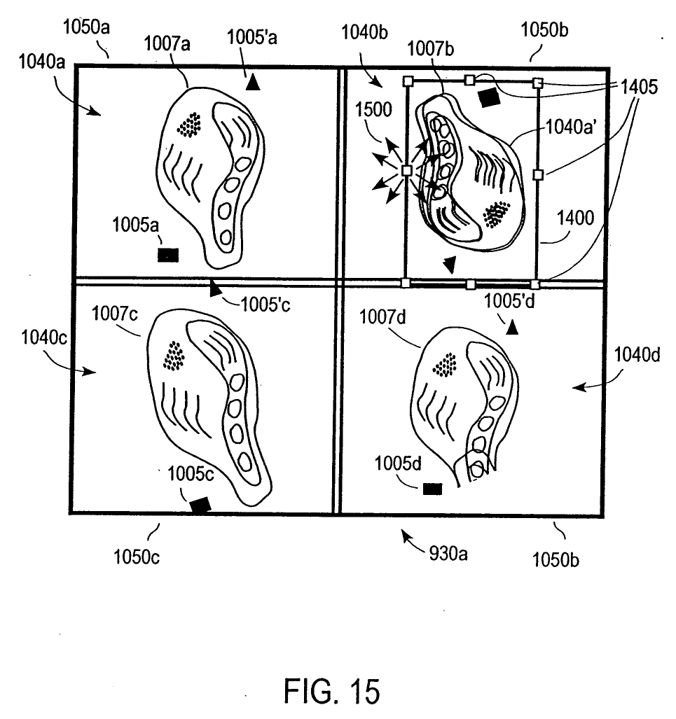

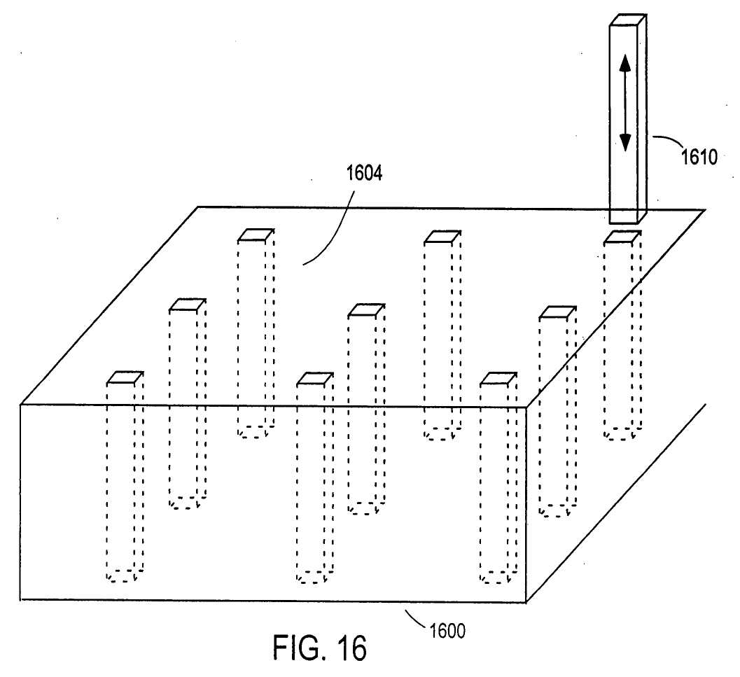

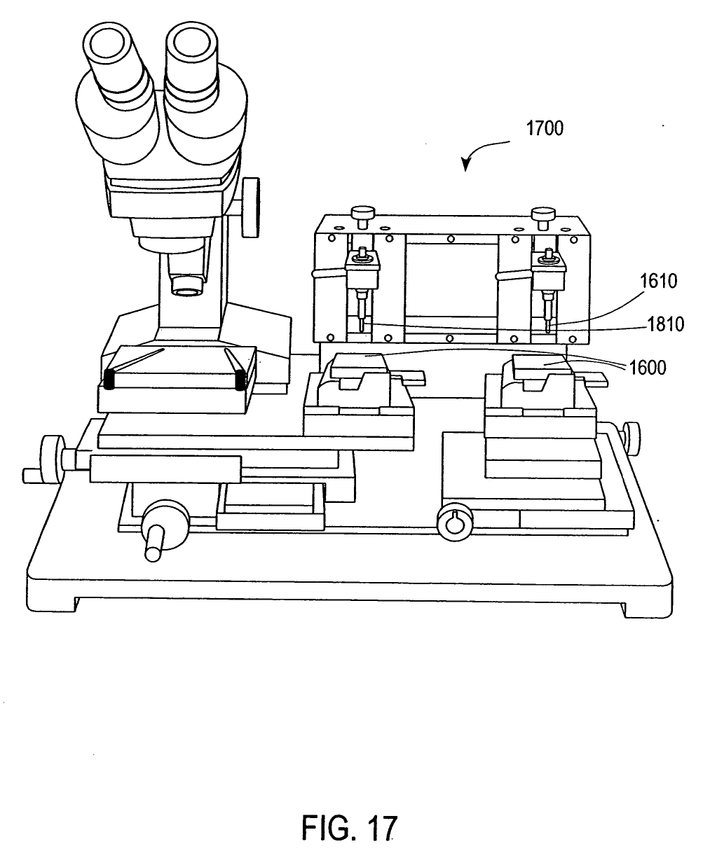

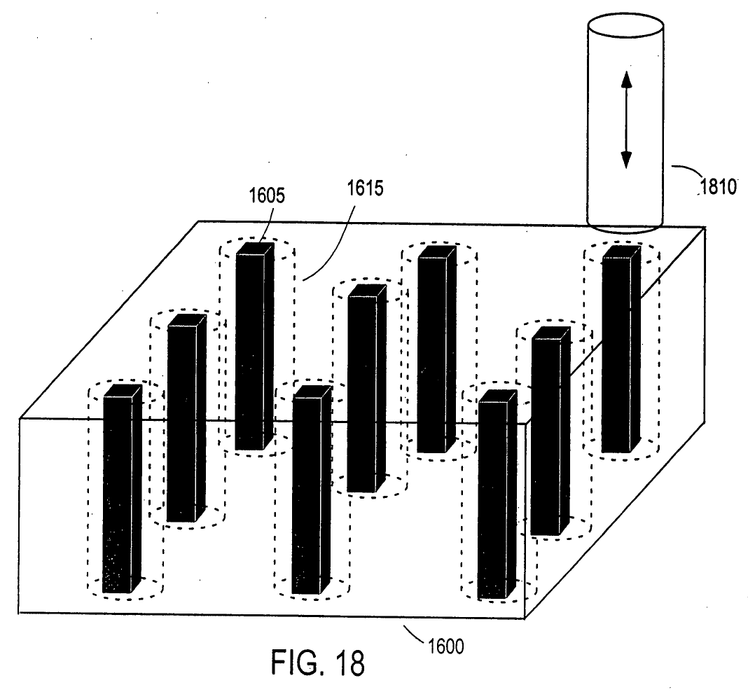

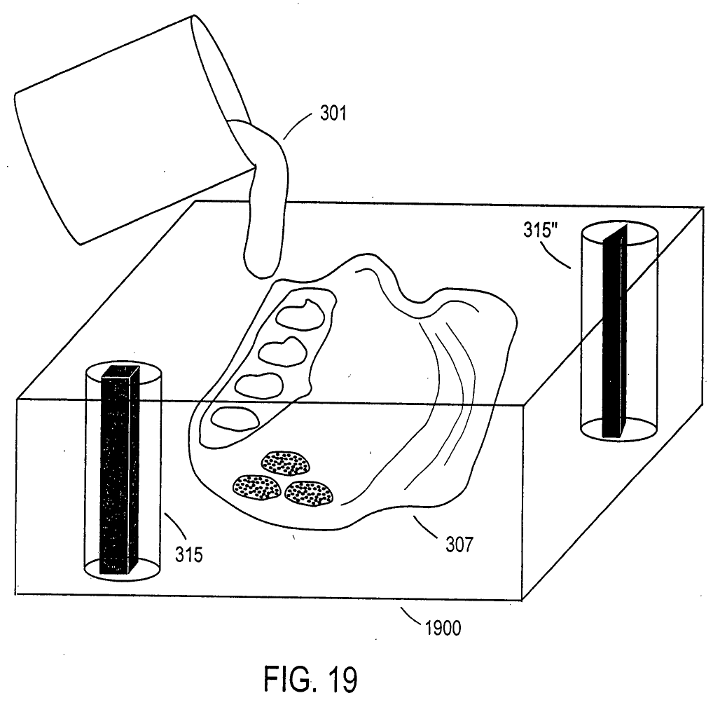

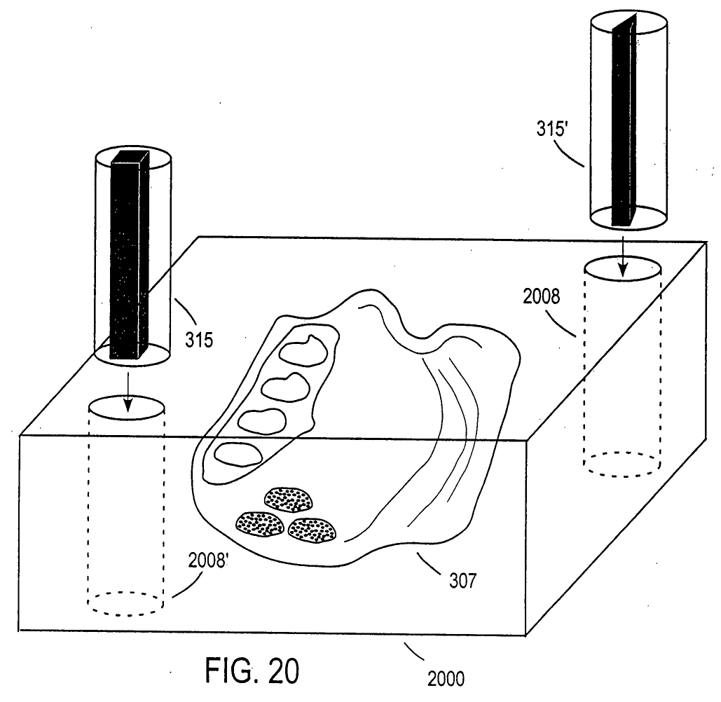

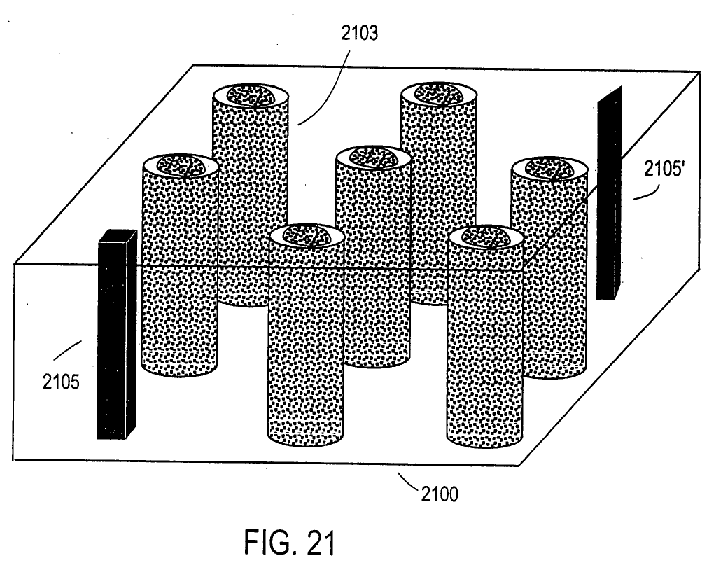

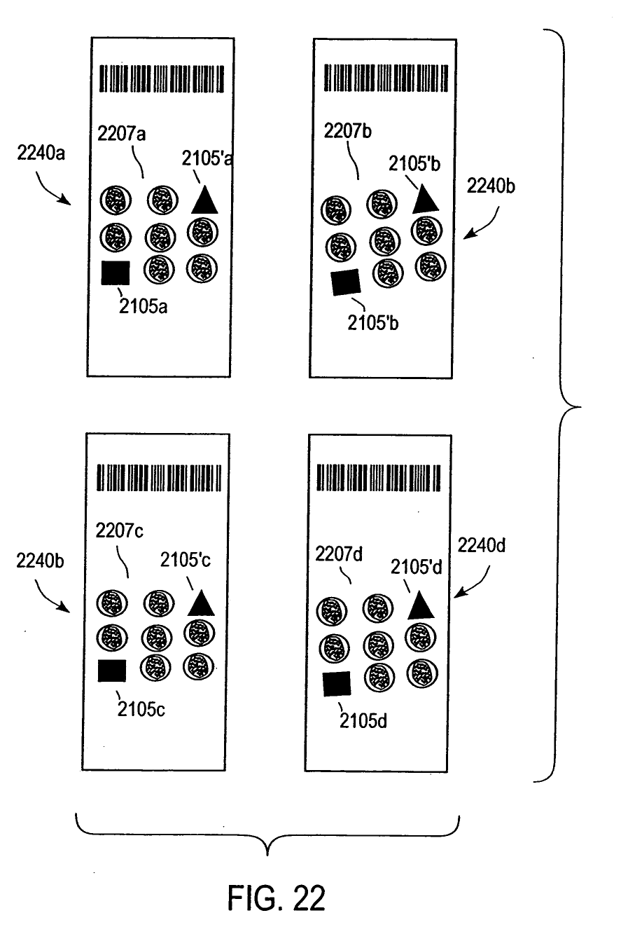

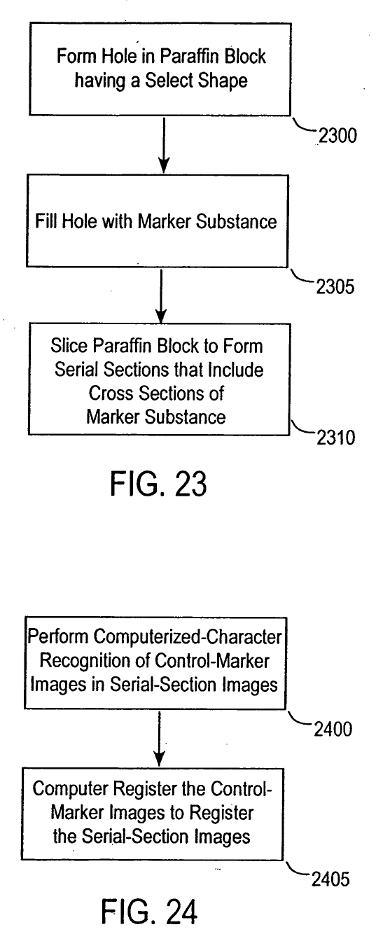

FIG. 10 is a simplified schematic of a set of serial-section images 1040 displayed on display 930a of system 900 and correspond, respectively, to serial sections 340a-340d. Each serial-section image includes a tissue-section image and control-marker images. In FIG. 10, each serial-section image is labeled with the base reference numeral 1007 and an alphabetic suffix. Also in FIG. 10, the control-marker images are labeled with the base reference numerals 1005 and 1005′ and alphabetic suffixes. Serial-section images 1040a-1040d are read from computer memory and displayed in screen windows 1050a-1050d, respectively. As described briefly above, system computer 930 is configured to register two or more serial-section images. To elaborate, computer 930 may be configured to register serial-section images using the control-marker images. According to one embodiment, computer 930 is configured to recognize the control-marker images, for example, using a pattern-recognition program running on the computer. Further, computer 930 is configured to discriminate between markers of different shapes, such as the rectangular and triangular shapes of control-marker images 1005 and 1005′. The computer further is configured to register the control-marker images once the control-marker images are recognized by the computer. That is, the computer is configured to shear, skew, rotate, and/or flip the serial-section images to register the control-marker images, and thereby, register the serial-section images.

According to one embodiment, a user can select two or more serial-section images the user would like the computer to register. The serial-section images might be selected by clicking on the serial-section images, dragging one serial-section image “over” another serial-section image, selecting the serial-section images from a tool bar, a menu (e.g., a drop down menu, a floating menu, etc.) or the like. A serial-section image that is dragged (or otherwise positioned) over another serial-section image is referred to as the ghost image, and the serial-section image that “underlies” the ghost image is referred to as the underlying image. A ghost image may be transparent, and the underlying image may be seen through the ghost image. Subsequent to selecting the serial-section images the user would like registered, computer 930 might execute the pattern-recognition program to recognize the control-marker images, and then register the control-marker images, thereby registering the serial-section images. The computer might be configured to display the selected serial-section images being registered. Specifically, the computer may position a ghost image over an underlying image and display the registration of the ghost image to the underlying image.

FIGS. 11A-11F show a time ordered sequence of events of computer registration of control-marker images 1005a and 1005′a to control-marker images 1005b and 1005′b. As shown in FIG. 10, serial-section image 1040b is compressed both vertically and horizontally with respect to serial-section image 1040a, and is rotated with respect to serial-section image 1040a. FIGS. 11A-11F show a process of skewing and rotating a ghost image 1040a′ (a copy of serial section image 1040a) to register control-marker images 1005a and 1005′a of the ghost image with the control-marker images 1005b and 1005′b of the underlying image 1040b. Note that underlying image 1040b may be viewed through the transparent ghost image 1040a′.

As described briefly above, the ghost image may be a transparent image, and the underlying image is visible through the ghost image. According to one embodiment, the transparency of the ghost image is adjustable to enhance the visualization of the ghost image or the underlying image. The transparency of the ghost image may be adjusted (e.g., from 0% to 100%) via a slider bar 1200 (or other technique) that is shown in FIG. 12. A transparency percentage 1205 of the ghost image may be indicated on the slider. For example, the ghost image in FIG. 12 is indicated as having a transparency of 80%. To indicate that the ghost image shown in FIG. 12 has a transparency of 80%, the ghost image is shown in phantom. According to one embodiment, the default transparency of the ghost image is 50%.

According to one embodiment, the ghost image and the underlying image are linked and locked (linking and locking are explained in detail below) such that graphical manipulation of one serial-section image causes each linked and locked serial-section image to be similarly manipulated. For example, magnifying one serial-section image, panning across the serial-section image, or rotating the serial-section image, causes respective magnifying, panning, or rotation of linked and locked serial-section images. Magnifying, panning, and/or rotating a set of serial-section images via the magnification, panning, and/or rotating of one serial-section image in the set provides for relatively rapid cross comparison of the serial-section images as magnifying, panning, and/or rotating do not need to be independently performed for each serial-section image. Magnification, panning, rotating or other graphical manipulations of serial-section images 1040a-1040d are controlled by a user using one or both input devices 930b and 930c. Graphical manipulations may be selected from drop-down menus, context menus, floating menus, graphical user interface (GUI) buttons displayed on display 930a, combinations of mouse clicks, combinations of mouse clicks and keyboard strokes, or other known computer control mechanisms.

According to one embodiment, serial-section images 1040a-1040d are mapped to a coordinate system 1300, which is shown superimposed on display 930c in FIG. 13. In mapping serial-section images to coordinate system 1300, image data, such as pixel-image data, of the serial-section images are assigned coordinates (e.g., (x,y) or (r,θ) coordinates) relative to their positions on coordinate system 1300. Coordinate system 1300 is used as a reference system to track the location of serial-section images and their associated image data, such as pixel-image data. Coordinates assigned to the pixel-image data are updated as the serial-section images are moved across display 930a and as the serial-section images are morphed to form transformed images. As referred to herein a transformed image is a serial-section image (e.g., a ghost image) that has been skewed, sheared, rotated, and/or flipped to register a set of control-marker images in the transformed image to another set of control-marker images in an underlying image.

While coordinate system 1300 is shown in FIG. 13 as an orthographic coordinate system (e.g., a Cartesian coordinate system), this is not necessary; coordinate system 1300 may be a polar coordinate system or other useful coordinate system. Further, while the origin of coordinate system 1300 is shown to be located in the lower left corner of display 930, the origin could be alternatively located.

According to some embodiments, subsequent to the registration of the control-marker images by computer 930, the registration of a ghost image and an underlying image may be manually refined.

As shown in FIG. 14, a reference-link region 1400 is positioned over a ghost image, such as ghost image 1040a′. The reference-link region includes a plurality of handles 1405 that may be selected and dragged to shear, skew, and/or rotate the ghost image. According to one embodiment, the handles are configured to move orthographically. According to an alternative embodiment, the handles can be moved in arbitrary directions to shear and skew a ghost image.

FIG. 15 shows a set of arrows 1500 that indicate a few of the arbitrary directions in which one of the handles 1405 can be moved. Each of the handles 1405 can be similarly moved in the directions indicated by arrows 1500, as well as numerous other directions. For a further understanding of the use of a reference-link region for shearing, skewing, and/or rotating a ghost image, see U.S. patent application Ser. No. ______ (Attorney Docket No. 016249-010200US) filed Jul. 28, 2004, titled “Linking of Images to Enable Simultaneous View of Multiple Objects,” and which is incorporated by reference herein in its entirety for all purposes.

Control-Marker Core, Control Marker, and Paraffin Block Formation

Control-marker cores may be formed by a variety of techniques. According to one embodiment of the present invention, control-marker cores are formed by introducing one or more marker substances (e.g., tissue, a tissue-like substance, a fluorescent material, ink, dye, a condensed material, etc.) into holes or apertures formed in a paraffin block, and filling the holes or apertures with liquid paraffin to surround the marker substances. Alternatively, a marker substance might be mixed with paraffin and poured or injected into a set of holes or apertures in a paraffin block to form one or more control-marker cores.

FIG. 16 is a simplified schematic of a paraffin block 1600 having a plurality of holes 1604 formed therein. The holes might be formed by inserting a coring device 1610 (e.g., a square needle) in the paraffin block to remove paraffin cores and thereby form the holes. The coring device might be hand operated or might be machine operated. For example, the coring device might be the needle of a tissue microarrayer that is configured to remove paraffin cores from paraffin blocks, and to insert cores of tissue samples into the formed holes to form a tissue microarray (TMA). Rather than inserting cores of tissues samples into formed holes of a paraffin block, a tissue microarrayer may be configured to fill the holes with a marker substance and paraffin.

FIG. 17 is a simplified schematic of a tissue microarrayer 1700 that might be configured to extract paraffin cores from a paraffin block in an array pattern, and might be configured to fill the holes with a marker substance to form, for example, an array of control-marker cores. Tissue microarrayer 1700 includes at least one coring device 1610 that is configured to remove paraffin cores from paraffin block 1600. The coring device might be a needle that has a shape that matches the shape of the control-marker cores to be formed. For example, the needle might have a shape that is rectangular, triangular, round or the like.

Subsequent to the formation of the plurality of holes 1604 in paraffin block 1600, the holes might be filled with a marker substance using coring device 1610 or other device to form control-marker cores 1605 shown in FIG. 18. For example, the tissue microarrayer might be configured to use coring device 1610 to fill one or more holes 1604 with a marker substance and paraffin. Alternatively, one or more holes 1604 might be filled by hand with a marker substance and paraffin, using for example coring device 1610. For example, coring device 1610 might be coupled to a syringe or other device to deposit a marker substance and paraffin into the holes.

Subsequent to the formation of control-marker cores 1605, one or more PCMCs 1615 (shown in FIG. 18 in phantom) may be cut from paraffin block 1600. The PCMCs might be cut from the paraffin block with a coring device 1810 (e.g., a round needle) that might have an inner diameter that matches the outer diameter of the PCMCs to be cut. For example the inner diameter of coring device might be about 1 millimeter—about 5 millimeters. Coring devices according to other embodiments of the present invention might have inner diameters that are less that 1 millimeter or greater than 5 millimeters. Coring device 1810 might be hand operated or machine operated to cut the PCMCs from the paraffin block. For example, tissue microarrayer 1700 might include coring device 1810 and might be configured to operate the coring device to cut the PCMCs from the paraffin block (see FIG. 17).

To form a paraffin block, such as paraffin block 300 that includes an embedded tissue sample 307 and control-marker cores 305 and 305′, PCMCs 315 and 315′ may be inserted in the paraffin block subsequent to the formation the paraffin block. Alternatively, the PCMCs may be positioned adjacent to the tissue sample as the tissue sample and the PCMCs are embedded in paraffin (e.g., as paraffin is poured into a mold that contains the tissue sample and the control markers).

FIG. 19 is a simplified schematic of a mold 1900 that has a tissue sample 307 and PCMCs 315 and 315′ disposed therein Paraffin 301 may be poured into mold 1900 to form paraffin block 300 shown in FIG. 3.

FIG. 20 is a simplified schematic of paraffin block 2000 having holes 2008 formed therein, for example, by a coring device. PCMCs 315 and 315′ might be inserted into holes 2008 with another coring device, such as coring device 1810 controlled, for example, by tissue microarrayer 1700. According to yet another alternative embodiment, holes may be formed in a paraffin block matching the shapes of control markers to be formed, and the holes may be filled with marker substance and paraffin to form the control-marker cores. One technique for forming holes in a paraffin block in the shape of a control marker and filling the holes with a marker substance and paraffin is generally shown in FIGS. 16 and 18 and is described above in detail.

According to one embodiment of the present invention, a TMA includes one or more control-marker cores. FIG. 21 is a simplified schematic of a TMA 2100 that includes a plurality of tissue-sample cores 2103 disposed in the array and includes first and second control-marker cores 2105 and 2105′ disposed at opposite corners of TMA 2100. While the control-marker cores 2105 and 2105′ are shown as being disposed at the corners of array 2105, the control-marker cores might be disposed at other locations in the TMA. Control-marker cores 2105 and 2105′ might be any of the control-marker cores described above in detail, such as control-marker cores 305, 305′, 405, 405′, 515, 515′ or the like. The control-marker cores might be embedded in the TMA according to one or more of the techniques described above, such as via the use of tissue microarrayer 1700, wherein one donor block of the tissue microarrayer is paraffin block 1600 or the like.

FIG. 22 is a simplified schematic of a set of serial sections 2240 that might be cut from TMA 2100. Each serial section includes a cross section of the tissue cores, labeled with reference numerals 2207a-2207d, and includes cross sections of the control-markers cores (or control markers that are labeled with reference numerals 2105a-2105d and 2105′a-2105′d).

FIG. 23 is a high-level flow chart having steps for generating a set of serial sections that includes tissue sections and control markers that have select shapes that provide positional information of the serial sections mounted on a set of slides. The control markers are further figured to stain to provide staining-control information for each serial section on each slide. The high-level flow chart is merely exemplary, and those of skill in the art will recognize various steps that might be added, deleted, and/or modified and are considered to be within the purview of the present invention. Therefore, the exemplary embodiment should not be viewed as limiting the invention as defined by the claims.

At 2300, at least one hole is formed in a paraffin block that includes a tissue sample substantially embedded therein. The hole has a select shape, such as rectangular, triangular, circular, an arbitrary and capricious shape or the like. At 2305, the hole is filled with a marker substance and paraffin. The marker substance and paraffin might be mixed in a substantially homogeneous mixture and inserted into the hole. At 2310, the paraffin block is sliced to form the set of serial sections. Each of the serial sections a tissue section and a control marker. Each control marker has the select shape. According to some embodiments, the serial sections are mounted, respectively, on a set of slides. During slicing, mounting, and processing (e.g., washing, staining, etc.) the serial sections can distort, rotate, and/or flip. The control markers are configured to distort, rotate, and/or flip in a substantially similar manner to substantially memorialize the distortions, rotations, and/or flipping of the serial sections.

FIG. 24 is a high-level flow chart having computerized steps for substantially registering a plurality of control-marker images that have select shapes and that are respectively included in a plurality of serial-section images, such that substantial registration of the shapes of the control-marker images provides for substantial registration of the serial-section images and of tissue-section images that might be included in the serial-section images. The high-level flow chart is merely exemplary, and those of skill in the art will recognize various steps that might be added, deleted, and/or modified and are considered to be within the purview of the present invention. Therefore, the exemplary embodiment should not be viewed as limiting the invention as defined by the claims.

At 2400, a computer is configured to perform pattern recognition on the control-marker images. At 2405, the select shapes of the control-marker images are registered, such that registration of the control-marker images registers the tissue-section images. According to one embodiment, the registration of the serial-section images is displayed on a display of the computer. According to yet another embodiment, registering the serial-section images includes at least one of shearing, skewing, rotating, and flipping at least one of the control-marker images to corresponding shear, skew, rotating, and flip the serial-section image associated with this control-marker image to register the control markers.

### CONCLUSION

It is understood that the examples and embodiments described above are for illustrative purposes only and that various modifications or changes in light thereof will be suggested to persons skilled in the art and are to be included within the spirit and purview of this application and scope of the appended claims. For example, while blocks, and control markers have been described as being formed at least in part from paraffin, blocks and control markers might be formed from other substances such as agar, resin, or the like. Further, while control markers are shown in the various figures as extending through paraffin blocks from top surfaces of the paraffin blocks to bottom surfaces of the paraffin block, according to some embodiments, the control markers may not extend to the surfaces of the paraffin blocks, but may be embedded within the paraffin blocks. Therefore, the above description should not be taken as limiting the scope of the invention as defined by the claims.

What is claimed is:

1. A sample configured to be cut to form a set of serial sections comprising:
a sample block;
at least one tissue sample substantially embedded in the sample block; and
a least one control-marker core substantially embedded in the sample block and having a select shape as viewed from an end of the control-marker core, wherein
each of at least two serial sections includes a cross section of the tissue sample and a cross section of the control-marker core,
each cross section of the tissue sample is referred to as a tissue section,
each cross section of the control-marker core is referred to as a control marker, and
each control marker has the select shape.

2. The sample according to claim 1, wherein the select shape is at least one of a square, a triangle, and a circle.

3. The sample according to claim 1, wherein if one of the serial sections is distorted, rotated, or flipped, the control marker associated with this serial section is configured to substantially similarly distort, rotate, or flip.

4. The sample according to claim 1, wherein at least one of the control markers is configured to stain as the serial section associated with this control marker is stained.

5. The sample according to claim 4, wherein the control marker that is configured to stain is configured to provide stain control information for the serial section that is associated with this control marker.

6. The sample according to claim 1, wherein registration of the select shapes of two or more of the control markers is configured to register the serial sections associated with these control markers.

7. The sample according to claim 1, wherein the sample block includes one of paraffin, agar, and resin.

8. The sample according to claim 1, wherein each control marker includes at least one of tissue, a tissue-like substance, a fluorescent material, ink, dye, and a condensed material.

9. The sample according to claim 8, wherein:
the tissue includes a control-cell line,
the fluorescent material includes one or more types of pollen grains,
the dye not soluble in water and alcohol, and
the condensed matter includes at least one of plastic, resin, and fibers.

10. The sample according to claim 1, wherein each control marker includes a plurality of marker layers, and wherein at least two of the layers include different marker substances.

11. The sample according to claim 10, wherein each marker layer includes at least one of tissue, a tissue-like substance, a fluorescent material, ink, dye, and a condensed material.

12. The sample according to claim 1, and further comprising at least a second control-marker core having a second select shape as viewed from an end of the second control marker.

13. The sample according to claim 12, wherein each serial section further includes a second cross section of the second control-marker core, each cross section of the second control-marker core is referred to as the second control marker.

14. A method of forming a set of serial sections comprising:
forming, in a paraffin block, at least one hole that has a select shape;
filling the hole with a marker substance; and
slicing the paraffin block to form the set of serial sections, wherein each of the serial sections includes a cross section of the tissue sample and a cross section of the marker substance, the cross sections of the tissue sample are referred to as the tissue sections, the cross sections of the marker substance are referred to as the control markers, and wherein each control marker has the select shape.

15. The method of claim 14, wherein the step of forming the hole further includes boring the hole with a needle that has the select shape.

16. The method of claim 15, wherein the needle is configured to be manually operated or operated by a tissue microarrayer.

17. The method of claim 14, wherein the step of forming the hole further includes boring the hole with a needle operated by a tissue microarrayer, and wherein the needle has the select shape.

18. The method of claim 14, and further comprising mounting the serial sections respectively on a set of slide, wherein at least one of the serial sections during mounting is distorted, rotated, or flipped, and the control marker associated with this serial section is substantially similarly distorted, rotated, or flipped.

19. The method of claim 14, wherein the select shapes of at least two of the control markers are configured to be registered to register the tissue sections associated with these control markers.

20. The method of claim 14, and further comprising at least one control markers memorializing at least one of a distortion, rotation, and flip of the serial section associated with this control marker.

21. The method of claim 14, and further comprising staining at least one of the serial sections, wherein the control marker associated with this serial section is configured to stain a select color, wherein the color the control marker stains is an indicator of whether this serial section correctly stains.

22. The method of claim 14, and further comprising staining each of the serial sections including staining the tissue sections and the control markers of the serial section, wherein control markers are configured to stain select colors, and the colors that the control markers stain are indicators of whether the tissue sections correctly stain.

23. A computerized method of registering a plurality of serial-section images that include tissue-section images and control-marker images comprising:
performing pattern recognition on the control-marker images, wherein the control marker images have one or more select shapes; and
registering the select shapes of the control-marker images to register the tissue-section images.

24. The method of claim 23, and further comprising displaying on a display the registering step.

25. The method of claim 23, wherein the step of registering includes at least one of shearing, skewing, rotating, and flipping at least one of the control-marker images to corresponding shear, skew, rotating, and flip the serial-section image associated with this control-marker image to register the control markers.

26. The method of claim 23, wherein the select shapes include at least one of a rectangle, a triangle, and a circle.

27. A set of serial sections cut from the sample of claim 1.

28. A set of serial sections formed by the method of claim 14.
